(19) United States
(12) Patent Application Publication
(10) Pub. No.: US 2024/0364138 A1
(43) Pub. Date: Oct. 31, 2024

(54) POWER TRANSFER ASSEMBLY AND AN ELECROSURGICAL INSTRUMENT

(71) Applicant: CREO MEDICAL LIMITED, Chepstow, Monmouthshire (GB)

(72) Inventors: Christopher Paul HANCOCK, Chepstow (GB); Malcolm WHITE, Chepstow (GB)

(21) Appl. No.: 18/291,793

(22) PCT Filed: Sep. 21, 2022
(86) PCT No.: PCT/EP2022/076155

§ 371 Date: Jan. 24, 2024

(30) Foreign Application Priority Data
Sep. 28, 2021 (GB) 2113836.7

(51) Int. Cl. H02J 50/10; A61B 18/00; A61B 18/12; A61B 18/14; A61B 18/18; H01B 9/00; H02J 50/00

(52) U.S. Cl. CPC H02J 50/10; A61B 18/1206; A61B 18/1482; A61B 18/1815; H01B 9/006; H02J 50/001; A61B 2018/00077; A61B 2018/00982; A61B 2018/1823; A61B 2018/1861

(57) ABSTRACT

Various embodiments provide a power transfer assembly. The assembly includes a transmitter comprising a coaxial cable having an inner conductor and an outer conductor separated by a dielectric material. The coaxial cable is configured to generate a magnetic field outside of the outer conductor when a first electromagnetic (EM) signal is conveyed by the coaxial cable. The assembly also includes a receiver for receiving power from the transmitter by inductive coupling using the magnetic field. Some other embodiments provide an electrosurgical instrument for delivering electromagnetic (EM) energy to biological tissue for tissue treatment. The instrument includes a feed structure, an applicator and the power transfer assembly.

### FIELD OF THE INVENTION

The present invention relates to a power transfer assembly including a coaxial cable. In particular, the power transfer assembly is configured to inductively couple power from the coaxial cable to a receiver when an electromagnetic signal is conveyed by the coaxial cable. Other embodiments include an electrosurgical instrument having the power transfer assembly.

### BACKGROUND TO THE INVENTION

It is often necessary to divert a signal from a cable, for example a coaxial cable, to power a secondary circuit. This is normally done using a filter, for example a diplexer or multiplexer.

However, there can be a high amount of loss in cables. Filters, such as diplexers and multiplexers can increase this loss, and therefore can decrease the efficiency of the cable when conveying a signal. Consequently, it may not be desirable to use a filter when trying to divert a signal from a cable to power a secondary circuit.

Furthermore, higher frequency signals, such as microwaves, are known to create high amounts of loss when being conveyed by coaxial cables. This is as energy from the higher frequency signal is lost to heating up the dielectric within the coaxial cable. As the loss when conveying higher frequency signals, such as microwaves, is higher, it may be even more important to avoid using apparatus such as filters to divert a signal from a cable, as to not increase the already higher amounts of loss.

### SUMMARY OF THE INVENTION

At its most general, the present invention discloses a power transfer assembly for wirelessly transferring power by inductive coupling using a coaxial cable. As the power transfer is wireless via inductive coupling, power is transferred from a transmitter to a receiver wirelessly or without a wired connection. For example, power may be transferred wirelessly through the air. This differs from wired transmission where power is transferred by a conductor. After wirelessly receiving power by inductive coupling, the receiver may be used to power a secondary circuit or an electrical element that requires power.

As the power transfer assembly enables wireless power transfer, the loss in the coaxial cable is not increased by using a wired structure such as a filter to divert a signal passing through the cable. Instead the power transfer assembly allows for wireless transfer of power using inductive coupling.

Inductive coupling enables wireless transfer as a power supply is wired to a primary coil and an oscillating current signal is sent through the primary coil which induces an oscillating magnetic field around the primary coil. The oscillating magnetic field induces an oscillating voltage signal in a secondary coil, placed close to the primary coil. In this way, electrical energy may be transmitted from the primary coil to the secondary coil by electromagnetic induction without the two coils being conductively connected. According to the invention, the primary coil is provided by the coaxial cable or a part thereof.

Using a coaxial cable for wireless power transfer has far reaching applications. For example, it may be desired to access energy being conveyed by a coaxial cable, without routing the signal being conveyed along the coaxial cable via a physical connection to a different circuit and consequently increasing the loss of the system. Instead, according to the invention, the energy being conveyed along the coaxial cable can be accessed via a wireless connection enabled by inductive coupling. Accordingly, the present invention has applications in telecommunications, safety, metering, medicine, electrosurgery, and any other application in which wireless power transfer using a coaxial cable is desired.

According to a first aspect of the invention there is provided a power transfer assembly comprising: a transmitter comprising a coaxial cable having an inner conductor and an outer conductor separated by a dielectric material, the coaxial cable being configured to generate a magnetic field outside of the outer conductor when a first electromagnetic (EM) signal is conveyed by the coaxial cable; and a receiver for receiving power from the transmitter by inductive coupling using the magnetic field.

Coaxial cables have an inner conductor and an outer conductor which are coaxial to each other and separated by a dielectric material. For coaxial cables, the magnetic field outside the cable is normally zero or near zero. This is as the signal conveyed by the inner conductor of the coaxial cable travels longitudinally (e.g. substantially in line with the cable between opposite ends of the cable) through the inner conductor, and longitudinally back through the outer conductor of the coaxial cable. Therefore, on a symmetrical coaxial cable with no defects, the magnetic field due to the signal conveyed by the inner conductor of the coaxial is cancelled (or near cancelled) by the signal conveyed by the outer conductor. This means that there is zero or near zero (e.g. negligible) magnetic field outside the outer conductor of the coaxial cable. For instance, any magnetic field present outside the outer conductor may be so minimal that it could not be used by a receiver for receiving a useful amount of power by inductive coupling. For example, a useful amount of power may be an amount of power required to operate an electric circuit, for example, an electric circuit comprising a microcontroller (MCU). For example, a useful amount of power may be at least 1 microwatt.

As a result, the magnetic field produced by the signal travelling down an inner conductor and outer conductor of a coaxial cable is confined to the dielectric that separates the inner and outer conductors, and to the conductors themselves.

However, when a coaxial cable is configured to generate a magnetic field outside of the outer conductor, the magnetic field generated by the signal conveyed by the outer conductor no longer cancels out (or near cancels out) the magnetic field generated by the signal conveyed by the inner conductor. This means that a magnetic field (e.g. a non-negligible magnetic field) is present outside the outer surface of the outer conductor of the coaxial cable. For example, a non-negligible magnetic field present outside the outer conductor may be large enough that it could be used by a receiver for receiving a useful amount of power by inductive coupling. For example, a useful amount of power may be an amount of power required to operate an electric circuit, for example, an electric circuit comprising a microcontroller (MCU). For example, a useful amount of power may be at least 1 microwatt.

The magnetic field (e.g. a non-negligible magnetic field) generated outside the outer surface of the outer conductor can be used for inductively coupling to by a receiver. The receiver may be connectable to a secondary circuit or electrical element to transfer power from the transmitter to the secondary circuit or electrical element.

The coaxial cable may be configured to generate the magnetic field (e.g. a non-negligible magnetic field) outside the cable when signals of a certain frequency or frequency range are conveyed down the cable. For example, the coaxial cable may be configured to generate a magnetic field (e.g. non-negligible magnetic field) outside of the outer conductor when a lower frequency signal (e.g. a radiofrequency (RF) signal) is conveyed by the coaxial cable. However, the coaxial cable may be configured not to generate a significant magnetic field (e.g. configured to generate a negligible magnetic field) outside of the outer conductor when a higher frequency signal (e.g. a microwave signal) is conveyed by the coaxial cable

The outer conductor may comprise a first helically wound conductor. The helically wound outer conductor may be leaky to produce a magnetic field (e.g. a non-negligible magnetic field) outside the outer conductor of the coaxial cable. For example, leakage on helically wound coaxial cables can be around 1/1,000^{th} to 1/10,000^{th} of the signal conveyed by the cable. As helically wound outer conductors can be leaky compared with other outer conductor types for coaxial cables, a helical wound outer conductor can be used for inductively coupling with a receiver to produce power within the receiver. Furthermore, using a helically wound outer conductor to produce a non-negligible magnetic field outside the coaxial cable can be advantageous. This is because as helically wound outer conductors can be leaky, they negate the need for physical modification of the coaxial cable to produce a magnetic field (e.g. a non-negligible magnetic field) outside the outer conductor. A possible reason for the leaky nature of helically wound conductors is that an amount of overlap between adjacent windings may vary along the length of the coaxial cable, for example, due to manufacturing tolerances. Additionally or alternatively, one or more gaps may be present between adjacent windings, again due to manufacturing tolerances.

The first helically wound conductor may comprise a defect configured to generate the magnetic field outside of the outer conductor. Helically wound conductors can contain defects that produce leakage of the non-negligible magnetic field of the coaxial cable. The defect may be any feature within a helically wound coaxial cable that causes the signal conveyed by the outer conductor not to cancel out (or not to near cancel out) the magnetic field produced by the signal on the inner conductor, and thus cause the coaxial cable to leak a non-negligible magnetic field i.e. for there to be a non-negligible magnetic field present outside of the outer conductor. The defect may exist in only a part (e.g. one discrete continuous region) or parts (e.g. more than one discrete continuous region) of the outer conductor. The defect may exist in a minority, a majority or an entirety of the outer conductor.

The defect may increase resistance between adjacent windings of the first helically wound conductor to cause the first EM signal to follow a helical path along the first helically wound conductor in a region local to the defect. When a signal is conveyed by a coaxial cable having a helically wound outer conductor, the signal normally follows a longitudinal path through the inner conductor and a longitudinal path through the outer conductor. If there is a defect causing an increase in resistance in a region, e.g. a region local to the defect, the signal may take a helical path through the region, e.g. a helical path along the first helically wound outer conductor rather than down the normal longitudinal path. This is because, due to the defect, the helical path may be a less resistive path in the defect's local region compared to the longitudinal path down the outer conductor in the defect's local region. For example, the overlap between adjacent windings of a helically wound outer conductor may have a greater resistance than a helical path around the conductor, and thus the first EM signal may take a helical path along the first helically wound conductor in a region local to the defect rather than longitudinally over the adjacent windings. The first EM signal may resume a longitudinal path outside the region local to the defect.

A helical path may be a path that travels helically around the outer conductor. For example, by following the helix of the helically wound conductor. Or, by taking a helical path around the outer conductor, where the helix of the helical path is different to the helix of the helically wound conductor. In contrast, a longitudinal path may be a path that travels longitudinally in line with the longitudinal axis of the coaxial cable. For 10 example, a path that travels longitudinally over adjacent windings of the helically wound conductor.

As the defect may cause the first EM signal to take a different, e.g. helical, path rather than the normal longitudinal path down the outer conductor, there is a disparity in paths between the helical path of the outer conductor and the longitudinal path of the inner conductor. Therefore, the magnetic field generated by the signal conveyed by the outer conductor no longer acts to cancel out (or near cancel out) the magnetic field generated by the signal conveyed by the inner conductor such that a magnetic field (e.g. a non-negligible magnetic field) appears outside the coaxial cable. This may cause the outer conductor to act as a solenoid in the region local to the defect, and radiate a magnetic field inside and outside the outer conductor of the coaxial cable. This magnetic field can be inductively coupled to by a receiver.

A defect may be any feature of the helically wound outer conductor that causes an increase in resistance in a region local to the defect. For example, the defect could be a section of poor contact between adjacent windings of a helically wound conductor, the poor contact causing an increase in resistance to cause the first EM signal to take a helical path along the first helically wound conductor in a region local to the defect. This is rather than the longitudinal path that the first EM signal would take if there was no defect. The result of the signal taking a helical path rather than a longitudinal path is that the magnetic field produced by signal conveyed by the outer conductor no longer cancels out (or near cancels out) the magnetic field produced by the signal on the inner conductor. Therefore, the coaxial cable produces a magnetic field (e.g. a non-negligible magnetic field) outside the outer conductor of the coaxial cable.

The defect may comprise a gap between adjacent windings of the first helically wound conductor. The adjacent windings of a helically wound conductor usually overlap to provide a continuous conducting surface. However, in a region there may be a gap between adjacent windings, e.g. due to an imperfect manufacturing process. The gap will cause an increase in resistance, resulting in the signal taking a helical path, rather than travelling longitudinally down the helically wound outer conductor. The gap may be between 1/10 microns and 10 microns. However, the gap may be any size that results in the first EM signal taking a helical path.

The outer conductor may further comprise a second helically wound conductor, wherein the first helically wound conductor has a first conductivity and the second helically wound conductor has a second conductivity, the first conductivity and the second conductivity being different to generate the magnetic field outside of the outer conductor. Having a difference in first conductivity and second conductivity can cause a region to have a higher resistance to the first EM signal travelling along a longitudinal path along the outer conductor, than a resistance to the first EM signal taking a helical path along the first helically wound conductor in a region local to the defect.

For example, if the first helically wound conductor has a higher resistance than the second helically wound outer conductor, at overlaps between adjacent windings of the helically wound conductors, the overlap may have a higher resistance compared with a path around the second helically wound conductor having a lower resistance. This can cause the signal to take an easier helical path through the region due to the increased resistance, rather than travelling longitudinally down the outer conductor.

The difference between the first conductivity and second conductivity may be caused by using different materials for the different helically wound conductors. For example, a first material may be used for the first helically wound conductor, and a second different material may be used for the second helically wound conductor. Common conductor materials may be silver, copper, gold and aluminium. However, other conductive materials may be used. Using a different material for the first and second helically wound conductors may produce a difference in conductivity between the different helically wound outer conductors.

However, the difference in conductivity between the first helically wound conductor and the second helically wound conductor could also be caused by varying other features, e.g. length or thickness, of the conductors to cause a difference between the first conductivity and the second conductivity.

The second helically wound conductor may also comprise a defect configured to generate the magnetic field (e.g. a non-negligible magnetic field) outside of the outer conductor. This defect may increase resistance between adjacent windings of the second helically wound conductor to cause the first EM signal to follow a helical path along the second helically wound conductor in a region local to the defect. In an embodiment, the above-described features of the defect of the first helically wound conductor are equally applicable to, and are hereby restated in respect of, the defect of the second helically wound conductor.

The principles discussed above in relation to helically wound outer conductors may also apply to braided outer conductors, or any other type of outer conductor, wherein the outer conductor has a non-uniformity that results in the magnetic field generated by the signal conveyed by the outer conductor not cancelling out the magnetic field generated by the signal conveyed by the inner conductor such that a non-negligible (or a useful) magnetic field remains outside the outer conductor. For example, a braided outer conductor, or other outer conductor type, e.g. foil, could have a defect or non-uniformity causing a non-negligible or useful magnetic field to be generated outside the outer conductor of the coaxial cable.

The transmitter may comprise an auxiliary conductor having a first portion and a second portion coupled to the outer conductor, the auxiliary conductor further having an intermediate portion between the first and second portions and which is spaced from the outer conductor to define a void, the auxiliary conductor and the outer conductor being configured to cause part of the first EM signal to flow through the auxiliary conductor.

As the first EM signal is conveyed by the outer conductor of the coaxial cable, a portion of the first EM signal will flow near the outer surface of the outer conductor. The auxiliary conductor offers an alternate path for the EM signal flowing near the outer surface of the outer conductor, rather than travelling down the outer conductor. For example, if part of the EM signal flows near the outer surface of the outer conductor, this part (or a portion thereof) may flow through the auxiliary conductor rather than the outer conductor.

The auxiliary conductor is coupled to the outer conductor at a first portion and a second portion. The first portion and the second portion may be a first end and a second end respectively. However, the first portion and the second portion could be any portion of the auxiliary conductor that is coupled to the outer conductor.

The intermediate portion is spaced apart from the outer conductor to define a void between the auxiliary conductor and the outer conductor. The separation of the intermediate portion to the outer conductor may be small compared with a wavelength of the first EM signal, for example being small compared with a wavelength of 750 m for a 400 kHz signal. The intermediate portion may be coupled or connected to the first portion or the second portion to allow a signal to be conveyed by the auxiliary conductor.

The auxiliary conductor conveys the first EM signal, and therefore at least part of the first EM signal will be conveyed by the auxiliary conductor rather than the outer conductor of the coaxial cable. Therefore, the entirety of the first EM signal will not be conveyed by the outer conductor (e.g. part is conveyed by the auxiliary conductor and the remaining part is conveyed by the outer conductor), and consequently the magnetic field generated by the signal conveyed by the outer conductor no longer cancels out (or no longer near cancels out) the magnetic field due to the signal on the inner conductor. This is as the signal flowing through the inner conductor will not wholly flow down through the outer conductor because part of it will flow through the auxiliary conductor. Therefore, the signal conveyed by the outer conductor no longer acts to cancel out (or no longer near cancels out) the magnetic field outside the coaxial cable. Thus, a magnetic field (e.g. a non-negligible magnetic field) will be generated outside the outer conductor. The magnetic field generated outside the outer conductor of the coaxial cable may be within the void, i.e. outside the outer surface of the outer conductor but inside the inner surface of the intermediate portion of the auxiliary conductor.

The EM signal will only flow through the auxiliary conductor at some frequencies. As the signal frequency increases, the skin depth of the signal decreases and so negligible current will be flowing near the outer surface of the outer conductor, meaning the auxiliary conductor will not convey the signal.

The auxiliary conductor may be any conductor which conveys an EM signal. For example, the auxiliary conductor may be a conducting bridge. The auxiliary conductor may be located coaxially around the outer conductor (i.e. around its whole circumference), for example enclosing the outer conductor. Or, the auxiliary conductor may be only located around a one or more circumferential portions of the outer conductor, for example the auxiliary conductor may be one or more longitudinal strips.

The transmitter may comprise an auxiliary conductor for conveying the EM signal when the outer conductor is helically wound. Or, the transmitter may comprise an auxiliary conductor when the power transfer assembly comprises an outer conductor is not helically wound.

The outer conductor may have a thickness and/or material to cause the first EM signal to flow through the auxiliary conductor. The thickness and/or material of the outer conductor may be set according to the skin depth of the signal being conveyed by the coaxial cable. That is, the thickness and/or material may be set so that, given the skin depth of the first EM signal, a portion of the first EM signal flows close to an outer surface of the outer conductor. The thickness and/or material may refer to the entirety of the outer conductor, or may refer only to a portion of the outer conductor, for example, a portion opposite or proximal to the receiver.

When an alternating current is passed through a conductor, a phenomenon known as the skin effect occurs. This is where conduction begins to move from an equal distribution over the entire cross section of a conductor to only existing at the surface of the conductor. As the frequency of the signal increases, the cross section of the area the signal flows through decreases. The skin depth of a signal is the distance within a conductor over which the current has fallen to 1/e compared to the value at the surface. It is also the thickness of the layer in which 1-1/e of the current flows, or about 63% of the current.

When an alternating current is passed through a coaxial cable, the signal flows near the outside surface of the inner conductor, and near the inside surface of the outer conductor. Therefore, in a coaxial cable, as the signal frequency increases, the current flowing through the outer conductor will move away from the outside surface of the outer conductor. Therefore, the thickness and/or material of the outer conductor may be set so that at some frequencies (e.g. RF frequencies) a significant proportion of the signal flows near the outer surface of the outer conductor. For example, the thickness and/or material may be set such that the thickness is between 1 and 5 skin depths of the first EM signal, resulting in a significant proportion of the first EM signal flowing near the outer surface of the outer conductor. At the same time, the thickness and/or material of the outer conductor may be set so that at some other frequencies (e.g. certain higher frequencies, such as microwave frequencies) a negligible or no proportion of the signal flows near the outer surface of the outer conductor.

The thickness of the outer conductor of the coaxial cable may be selected so that thickness results in a significant portion of the first EM signal flowing near the surface of the outer conductor. For example, 0. 1 mm can be considered to be a reasonably thick outer conductor, but at some lower frequencies, e.g. RF frequencies such as 400 kHz, this will be less than one skin depth thick, and therefore the current flowing near the outer surface of the outer conductor will not have fallen to even 37% of the current flowing at the inner surface of the outer conductor. Thus, a significant portion of the signal conveyed by the outer conductor will be flowing near the outer surface of the outer conductor. In an embodiment, the outer conductor may be less than 0.25 mm thick.

On a coaxial cable that is not configured to generate a magnetic field (e.g. a non-negligible or useful magnetic field) outside of the outer conductor, the magnetic field outside the coaxial cable is zero or near zero (e.g. negligible, unuseful). As the magnetic field outside the coaxial cable is zero or near zero, the current at the outer surface of the outer conductor must also be zero or near zero. This results in a denser current distribution in the outer conductor, than would occur in a conductor with a thickness significantly thicker than the skin depth of the signal flowing through the conductor. As the current distribution is denser and thus more resistant to current flow, when an auxiliary conductor is coupled to the outer conductor at least part of a first EM signal (e.g. a non-negligible part or a useful part) flowing through the outer conductor will flow through the easier path of the auxiliary conductor. This is because the value of the current or signal is zero (or near zero) at the surface, until the auxiliary conductor is introduced. Introducing the auxiliary conductor makes the value of the current or signal at the surface non-zero as the signal flows through the auxiliary conductor. The non-zero value when the auxiliary conductor is connected enables the signal to flow through the auxiliary conductor.

Therefore, at least part of the signal will flow through the auxiliary conductor when the auxiliary conductor is coupled to the outer conductor. For example, the thickness of the outer conductor may be set to have a skin depth correlating to one skin depth of the first EM signal. This means that the first EM signal would have only fallen to 1/e of the current flowing on the inside surface of the outer conductor. However, the thickness of the outer conductor may be set to any ratio of thickness of the outer conductor to skin depth of an EM signal, to cause the signal to have a high enough current density near the outer surface of the outer conductor, so that a portion of the EM signal flows through the auxiliary conductor rather than the outer conductor.

The skin depth of a signal is different in different materials. Therefore, the material of the outer conductor may be selected to cause the signal to have a high enough current density near the outer surface of the outer conductor, so that the EM signal flows through the auxiliary conductor.

Table 1 provides values of skin depth at spot frequencies of 5.8 GHZ and 400 kHz for commonly used conductive materials. This table illustrates the different values of skin depth for different materials and frequencies.

TABLE 1
Skin depth (in μm) for various commonly used
materials at frequencies of 400 kHz and 5.8 GHz.

| | Electric | Electric | | |
|---|---|---|---|---|
| | conductivity | resistivity | Skin depth | |
| | (10.E6 | (10.E−8 | (μm at frequency) | |
| Material | Siemens/m) | Ohms · m) | 400 kHz | 5.8 GHz |
| Silver | 62.1 | 1.6 | 178.99 | 1.49 |
| Copper | 58.7 | 1.7 | 184.10 | 1.53 |
| Gold | 44.2 | 2.3 | 212.16 | 1.76 |
| Aluminium | 36.9 | 2.7 | 232.19 | 1.93 |

The outer conductor may comprise a thinned section and the auxiliary conductor may be coupled to the thinned section, the thinned section having a thickness to cause the first EM signal to flow through the auxiliary conductor. A section of the outer conductor may be thinned to create a thinned section, e.g. the thinned section of the outer conductor may be thinner than some or all other sections of the outer conductor. The thinned section may be thinned so that a significant portion of the EM signal conveyed by the outer conductor flows near the outer surface of the outer conductor, so that when the auxiliary conductor is coupled to the thinned section the EM signal flows through the auxiliary conductor. The other sections of the outer conductor may have an increased thickness compared to the thinned section. These sections may be many skin depths deep, so that the first EM signal does not flow near the outer surface of the outer conductor in these other sections. Therefore, the thinned section may act like a coupling section of the outer conductor, to have a thinner thickness to allow the first EM signal to flow through the auxiliary conductor.

However, even in embodiments where the outer conductor has a uniform thickness along its length, and where the thickness is thin enough to result in the first EM signal flowing near the outer surface along its whole length, the magnetic field (or useful, non-negligible magnetic field) would only be produced outside the outer conductor in a section of the outer conductor that is configured to generate a magnetic field outside the outer conductor, e.g. a section having an auxiliary conductor or a magnetic field generating defect.

This allows the coaxial cable to be configured to generate a non-negligible magnetic field outside the outer conductor, by thinning only one section, rather than selecting a thickness for the whole outer conductor. Consequently, it may be possible to modify existing coaxial cables to generate a non-negligible magnetic field outside the outer conductor by thinning a section of the outer conductor.

The thinned section will not affect higher frequency signals where the skin depth of the higher frequency is many times smaller than the thickness of the thinned section. This is because due to the skin depth effect negligible current from the higher frequency signal will be flowing near the outer surface of the thinned section.

The first portion and/or the second portion may be physically coupled to the outer conductor. For example, the first portion and/or the second portion may be directly connected to the outer conductor. Or, there may be one or more intervening elements, e.g. other conductors, located in between, i.e. the connection may be indirect. The physical coupling conveys the EM signal to cause the EM signal to flow through the auxiliary conductor. The physical coupling may be a soldered or a touching/contact connection.

The first portion and/or the second portion may be spaced apart from the outer conductor to form a capacitive element for capacitively coupling the auxiliary conductor to the outer conductor. For example, the first portion and/or the second portion may be spaced apart from the outer conductor, and the first EM signal may be conveyed by capacitance to flow through the auxiliary conductor. The capacitive element may be a capacitor, or any capacitive element that capacitively couples the auxiliary conductor to the outer conductor. The first portion may be capacitively coupled to the outer conductor, and the second portion may be physically coupled to the outer conductor. Or, the second portion may be capacitively coupled and the first portion physically coupled. Both portions may be capacitively coupled. A capacitive connection may enable an EM signal to be conveyed by the auxiliary conductor when a physical, e.g. soldered or touching connection, is not possible or desired.

The receiver of the power transfer assembly may be located in the void. A magnetic field (e.g. a non-negligible magnetic field) may be produced in the void between the outer conductor and the auxiliary conductor (e.g. intermediate portion). The receiver may be located within this void to interact with the magnetic field within the void. The magnetic field may be advantageously strongest in the void, and may be optimised for inductive coupling by choosing an optimal thickness and/or material (e.g. conductivity) of the outer conductor, and optimal thickness and/or material of the auxiliary conductor. An optimal thickness and/or material refers to a thickness and/or material which produces a strong magnetic field in the void.

The receiver may comprise a magnetizable element. The magnetizable element is any element that is capable of being excited by the non-negligible magnetic field. For example, the magnetizable element may be an element with magnetic properties. The magnetizable element may be an element having a high magnetic permeability. The magnetizable element advantageously increases and concentrates the magnetic field that links the transmitter and receiver. The magnetizable element may be ferrous or comprise a ferrite element. For example, the magnetizable element may comprise a ferrite or iron core.

The receiver may further comprise a receiving conductor wound around the magnetizable element. The receiving conductor is inductively coupled to the transmitter, such that the magnetic field (e.g. a non-negligible magnetic field) generated outside the outer conductor by the coaxial cable induces a current within the receiving conductor enabling a wireless transfer of power from the transmitter to the receiver. The receiving conductor may be wound around the magnetizable element. The receiving conductor may be wound into a helix or a coil. For example, the receiving conductor may comprise a solenoid. A receiving conductor wound around the magnetizable element advantageously increases coupling strength between the transmitter and receiver. The receiving conductor may be connected to, and power, a secondary circuit or an electrical element.

The magnetizable element may comprise a toroid. A toroid is a surface of a revolution with a hole in the middle. The toroid may be ring or doughnut shaped. Toroid shaped magnetizable elements have superior electrical performance and efficiency. The toroid shaped magnetizable element may be a toroid shaped magnetic core.

The outer conductor may be configured not to generate a magnetic field outside of the outer conductor when a second EM signal is conveyed by the coaxial cable, the second signal having a higher frequency than the first signal.

The second EM signal travels through the inner conductor and outer conductor of the coaxial cable like the first EM signal, however when the second EM signal is conveyed by the coaxial cable the coaxial cable does not generate a magnetic field (or useful, i.e. non-negligible magnetic field) outside of the outer conductor. Therefore, coupling to the lower frequency first EM signal can be done using the outer conductor that completely shields the higher frequency second EM signal, for all practical purposes.

The skin depth of a signal varies with the frequency of the signal. As such, higher frequency signals have a much smaller skin depth than lower frequency signals. For example, a 5.8 GHz signal has a skin depth of 1.49 μm when conveyed by a silver conductor. If the outer conductor has a thickness of 100 μm or 0.1 mm, the outer surface of the outer conductor will be roughly 67 skin depths thick. Therefore, the current levels at the outer surface of the outer conductor will be, for example, (1/e) \hat{ }67 or 7.985e-30 of the levels at the inner surface of the outer conductor. This means that at high frequencies, negligible current will flow near the outer surface of the outer conductor.

Therefore, the second EM signal would not be conveyed by an auxiliary conductor coupled to the outer conductor, as negligible current flows near the outer surface of the outer conductor. This means that the magnetic field generated by the second EM signal conveyed by the outer conductor cancels out (or near cancels out) the magnetic field produced by the second EM signal conveyed by the inner conductor. Thus, for the second EM signal, zero or near zero magnetic field is produced outside the outer conductor of the coaxial cable, as the coaxial cable is not configured to generate a useful or non-negligible magnetic field outside the coaxial cable when conveying a second EM signal.

Leakage on helically wound conductors is significantly lower when conveying higher frequency signals. This is because capacitive susceptance is higher when higher frequency signals are conveyed, and the skin depth of higher frequency signals is lower. For example, when a 400 kHz signal is conveyed across a defect, the capacitive susceptance of the defect is roughly 10,000 times less than the capacitive susceptance of the defect when a signal at 5.8 GHz is conveyed. Therefore, the second EM signal travels substantially via a longitudinal path down the outer conductor, despite any defects on the helically wound conductor. This is different to the first EM signal, wherein the defects of the helically wound conductor cause the first EM signal to take a helical path rather than a longitudinal path. As the defects do not result in the second EM signal taking a helical path, the magnetic field generated by the second EM signal flowing in the outer conductor cancels out (e.g. substantially or completely) the magnetic field generated by the second EM signal flowing in the inner conductor. Therefore, a magnetic field (e.g. a non-negligible or useful magnetic field) is not generated outside the outer conductor when the helically wound outer conductor conveys a second EM signal, as the coaxial cable is not configured to generate a magnetic field outside the coaxial cable when conveying a second EM signal.

The outer conductor being configured not to generate a magnetic field outside of the outer conductor when a higher frequency second EM signal is conveyed by the coaxial cable, advantageously allows the receiver to couple to the first EM signal and not the second EM signal. Higher frequency EM signals can have significant loss when being conveyed in coaxial cables. Therefore, by using only the lower frequency first EM signal for inductively coupling with the receiver, the higher frequency second EM signal is left untouched. Consequently, the power transfer assembly creates little or no loss for the higher frequency second EM signal. Only the first EM signal is used for coupling to. This allows the outer conductor of the coaxial cable to act almost as a “filter” but without the increased loss within the coaxial cable that would result from using an actual filter.

The second EM signal may be a microwave (MW) signal. In this specification “microwave” or “MW” may be used broadly to indicate a frequency range of 400 MHZ to 100 GHz. A specific frequency that has been considered is 5.8 GHZ.

The first EM signal may be a radiofrequency (RF) signal. In this specification “radiofrequency” of “RF” may be used to broadly indicate a frequency range of up to 300 MHz, preferably 10 kHz to 1 MHz. A specific frequency that has been considered is 400 KHz.

The outer conductor may have a thickness greater than 8 microns. However, in the above-described embodiments in which a gap exists between adjacent windings of a helically wound conductor, the thickness of the outer conductor in the gap would be zero. This thickness may enable negligible current from the second EM signal to be flowing near the outer surface of the outer conductor. This thickness may enable a significant portion of current from the first EM signal to be flowing near the outer surface of the outer conductor. Thus, a magnetic field (e.g. useful or non-negligible magnetic field) may be generated outside the outer conductor when the outer conductor having a thickness of greater than 8 microns conveys the first EM signal.

According to a second aspect of the invention there is provided an electrosurgical instrument for delivering electromagnetic (EM) energy to biological tissue for tissue treatment, comprising: a feed structure and an applicator located at a distal end of the feed structure, the feed structure for conveying the EM energy from an energy source to the applicator, the applicator for delivering the EM energy into biological tissue at a treatment site; and a power transfer assembly according to the first aspect, wherein the feed structure comprises the coaxial cable of the transmitter and the EM energy comprises the first EM signal. In an embodiment including the second EM signal, the EM energy may also include the second EM signal.

An electrosurgical instrument may be any instrument for electrosurgery. For example, any instrument utilising electrical current or EM energy to cut, ablate, coagulate, treat or cauterise tissue. The feed structure conveys energy from an energy source to an applicator. The feed structure may be a coaxial cable, or may comprise a coaxial cable along with other conductor types.

The energy source may be any suitable energy source for generating EM signals. For example, the energy source may generate a first EM signal, which may be an RF signal. The energy source may also generate a second EM signal, which may be a MW signal. The energy source may be a generator. The energy source may be connected to a proximal end of the feed structure by a suitable connector, e.g. a QMA connector.

The applicator may be any suitable structure for delivering EM energy to biological tissue for tissue treatment. For example, the applicator may comprise an antenna for delivering EM energy. The applicator may comprise a first electrode and a second electrode for delivering EM energy, wherein the first electrode is connected to the inner conductor of the coaxial cable and the second electrode is connected to the outer conductor of the coaxial cable. The applicator may cut, ablate, coagulate, treat or cauterise tissue.

The electrosurgical instrument comprises a power transfer assembly according to the first aspect. The power transfer assembly may be any power transfer assembly disclosed in this specification. The power transfer assembly comprises a transmitter comprising a coaxial cable having an inner conductor and an outer conductor separated by a dielectric material, the coaxial cable being configured to generate a magnetic field outside of the outer conductor when a first electromagnetic (EM) signal is conveyed by the coaxial cable; and a receiver for receiving power from the transmitter by inductive coupling using the magnetic field. The power transfer assembly may be used for wirelessly transferring power from the feed structure of the electrosurgical instrument to a receiver. The power transfer assembly can be located at any point along the feed structure. The power transfer assembly may be located in a structure located around the feed structure.

The feed structure of the electrosurgical instrument comprises the coaxial cable of the transmitter. For example, a section of the feed structure may be the coaxial cable of the transmitter. Or, the whole feed structure may be a coaxial cable, of which part is a transmitting section.

The feed structure may comprise other conductive elements to convey energy from an energy source to an applicator.

The receiver of the power assembly may be located proximal to the transmitter to receive power from the transmitter for inductive coupling using the magnetic field. The receiver may be integrated into the feed structure. Or the receiver may be located within another structure proximal to the feed structure to allow for inductive coupling to the transmitter. The receiver may be located within a structure located around the feed structure. The receiver of the power transfer assembly may be used to power a secondary circuit. The secondary circuit may be used to power an electrical element for use with electrosurgery or otherwise.

The electrosurgical instrument may be sized for insertion through an instrument channel of a surgical scoping device. For example, the electrosurgical instrument may be dimensioned to fit within a surgical scoping device. A surgical scoping device may be for example, a laparoscope or an endoscope. The electrosurgical instrument being for a surgical scoping device allows the electrosurgical instrument to advantageously be used for minimally invasive tissue treatment within hard to reach internal parts of a patient's body, e.g. within the lungs, pancreas, liver, kidneys or gastrointestinal tract.

The invention includes the combination of the aspects and preferred features described except where such a combination is clearly impermissible or expressly avoided.

### SUMMARY OF THE FIGURES

Embodiments illustrating the principles of the invention will now be discussed with reference to the accompanying figures in which:

FIG. 1 is a schematic diagram of a power transfer assembly with an auxiliary conductor according to a first embodiment;

FIG. 2 is a schematic cutaway view of the power transfer assembly of FIG. 1;

FIG. 3 is a first cross section view of the power transfer assembly of FIG. 1;

FIG. 4 is second cross section view of the power transfer assembly of FIG. 1;

FIG. 5 is a schematic diagram of a power transfer assembly with a helically wound conductor according to a second embodiment;

FIG. 6 is a schematic diagram of a power transfer assembly with an auxiliary conductor and helically wound outer conductor according to a third embodiment; and

FIG. 7 is an electrosurgical system comprising a power transfer assembly according to a fourth embodiment.

### DETAILED DESCRIPTION OF THE INVENTION

Aspects and embodiments of the present invention will now be discussed with reference to the accompanying figures. Further aspects and embodiments will be apparent to those skilled in the art. All documents mentioned in this text are incorporated herein by reference. Where features of the embodiments described below are equivalent, the same reference numerals are used and detailed description thereof is not repeated.

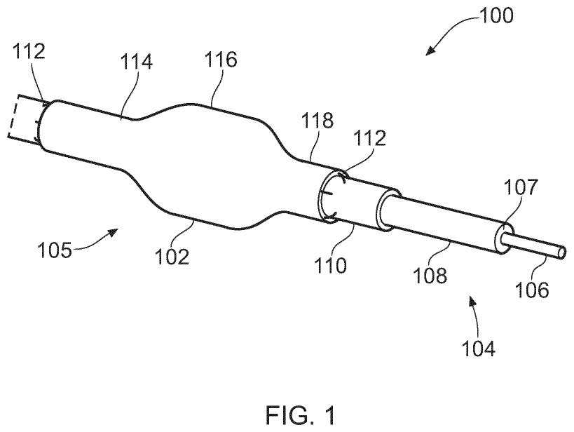

FIG. 1 shows a schematic diagram of a power transfer assembly 100 with an auxiliary conductor 102 according to a first embodiment. The power transfer assembly 100 comprises a transmitter 105 comprising a coaxial cable 104. The coaxial cable 104 has an inner conductor 106 and an outer conductor 108. The inner conductor 106 and the outer conductor 108 are separated by a dielectric 107. The coaxial cable 104 may comprise a protective jacket 110 located around the outer conductor 108 to protect the coaxial cable 104 and, perhaps, to keep the outer conductor 108 together. However, in some embodiments the coaxial cable 104 may not have a protective jacket 110. However, in some embodiments the coaxial cable 104 may not have a protective jacket 110. In this embodiment, the transmitter 105 further comprises an auxiliary conductor 102 coupled to the outer conductor 108. In this embodiment, the auxiliary conductor 102 is a conducting bridge. The auxiliary conductor 102 is coupled to the outer conductor 108 at a first portion 114 and, separately, at a second portion 118. An intermediate portion 116 is between the first portion 114 and the second portion 118. In this embodiment, auxiliary conductor 102 is coupled to the outer conductor 108 using physical electrical contacts 112. In embodiments with a protective jacket 110, the electrical contacts 112 pierce the jacket to make electrical contact with the outer conductor. In embodiments without a protective jacket 110, the electrical contacts 112 may be present or, alternatively, the auxiliary conductor 102 may be directly connected (e.g. soldered) to the outer conductor such that no electrical contacts 112 are required. However, in some other embodiments the connection may be capacitive.

The coaxial cable 104 is configured to generate a magnetic field (e.g. a non-negligible magnetic field) outside of the outer conductor 108 when conveying a first EM signal, as the coaxial cable 104 is connected to the auxiliary conductor 102. The first EM signal may be a RF signal. In this embodiment, the coaxial cable 104 has an auxiliary conductor 102 coupled to the outer conductor 108. Therefore, when the first EM signal travels down the outer conductor 108, at least part of the first EM signal travels through the auxiliary conductor 102. This is because the auxiliary conductor 102 may have a thickness and/or material selected so that a portion of the first EM signal flows near the outer surface of the outer conductor 108, and therefore flows through the auxiliary conductor 102. When a higher frequency second EM signal flows through the coaxial cable 104, the second EM signal is not conveyed by the auxiliary conductor 102 as the second EM signal has a higher frequency. Specifically, due to the skin depth effect, for the second EM signal, zero or negligible current flows near the outer surface of the outer conductor 108. Consequently, zero (or near zero) magnetic field is generated outside the coaxial cable 104 when the coaxial cable 104 conveys the second EM signal. The second EM signal may be a MW signal.

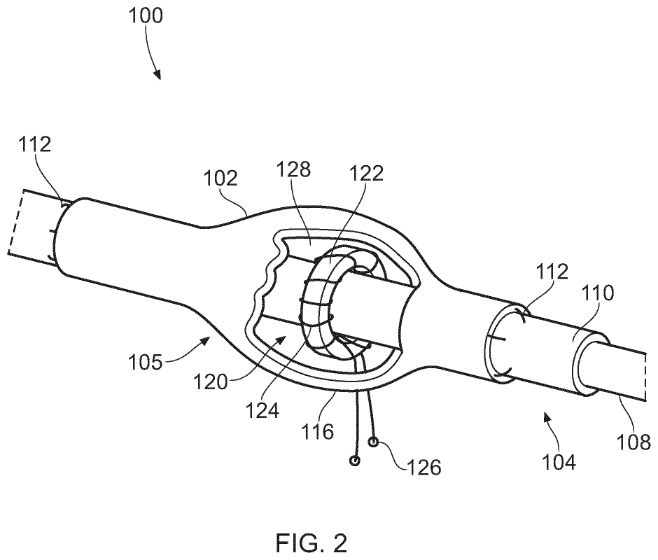

FIG. 2 shows a schematic cutaway view of the power transfer assembly 100 of FIG. 1. FIG. 2 shows a cutaway view, exposing the inside of the auxiliary conductor 102. The intermediate portion 116 is spaced apart from the outer conductor 108 to define a void 128. Inside the void 128 is a receiver 120. The receiver 120 comprises a magnetizable element 122, and a receiving conductor 124. In this embodiment, the magnetizable element 122 is a ferrite core, and the receiving conductor 124 is a secondary winding. The receiving conductor 124 is connectable at ends 126 to another electrical element or a secondary circuit.

When the first EM signal conveyed by the coaxial cable 104 travels through the outer conductor 108, at least part of the first EM signal will travel through the auxiliary conductor 102. This creates a magnetic field outside the outer conductor 108 and within the void 128. The receiver 120 is located within the void 128, and therefore receives power via inductive coupling from magnetic field generated by the transmitter 105. This generates a current within receiving conductor 124, and this can be used to power an electrical element or secondary circuit connected to ends 126.

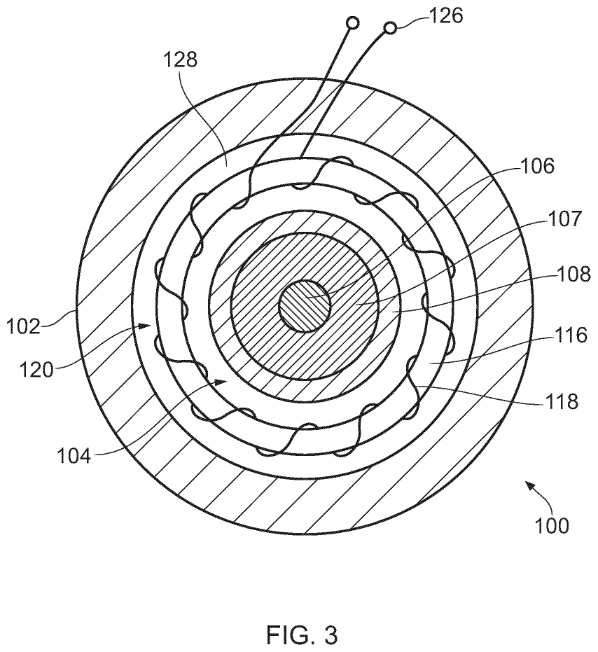

FIG. 3 shows a first cross section view of the power transfer assembly 100 of FIG. 1. The protective jacket 110 is not shown in this cross-sectional view for clarity. The first cross section view is taken along a line through the auxiliary conductor 102 and perpendicular to a longitudinal axis of the coaxial cable. As shown in FIGS. 2 and 3, the receiver 120 is located in the void 128 between the outer conductor 108 of the coaxial cable 104 and the auxiliary conductor 102 (e.g. the intermediate portion 116). The ends 126 of the receiving conductor 124 are connectable to an electrical element outside the power transfer assembly 100. In this embodiment, the receiver 120 and the auxiliary conductor 102 are shown encircling the coaxial cable 104.

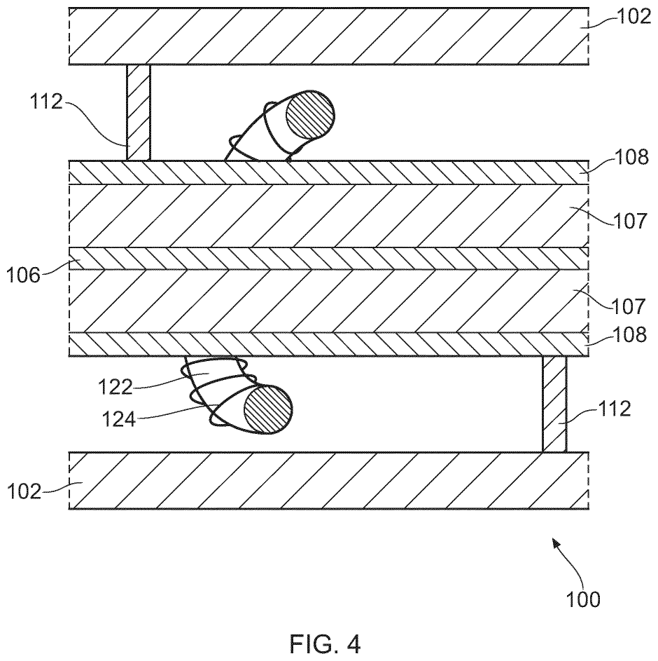

FIG. 4 shows a second cross section view of the power transfer assembly of FIG. 1. The protective jacket 110 is not shown in this second cross-sectional view for clarity. The second cross section view is taken along a line through the auxiliary conductor 102 and in line with the longitudinal axis of the coaxial cable. The auxiliary conductor 102 is shown connected to the outer conductor 108 of the coaxial cable 104 using electrical contacts 112. For example, the left contact 112 may connect the first portion 114 to the outer conductor 108, whereas the right contact 112 may connect the second portion 118 to the outer conductor 108. Although FIG. 4 only shows one electrical contact 112 on each side, there may be a plurality of contacts on each side.

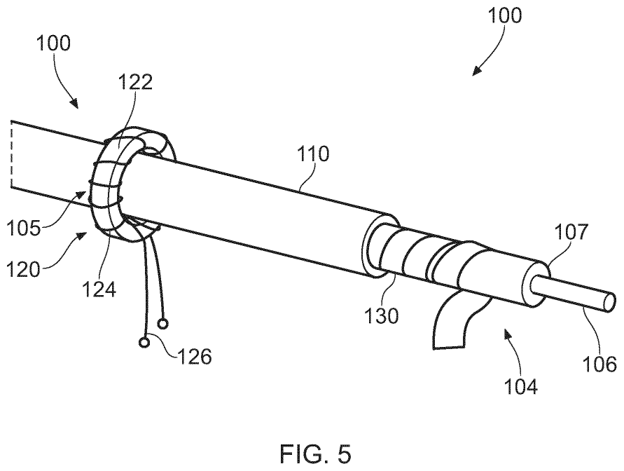

FIG. 5 shows a schematic diagram of a power transfer assembly 100 with a helically wound conductor 130 according to a second embodiment. This embodiment does not have the auxiliary conductor 102. As such, in this embodiment, the transmitter 105 comprises a coaxial cable 104 having a helically wound outer conductor 130.

The coaxial cable 104 is configured to generate a magnetic field (e.g. a non-negligible magnetic field) outside the coaxial cable 104 when a first EM signal is conveyed by the coaxial cable 104, as the coaxial cable 104 has a helically wound outer conductor 130. The first EM signal may be a RF signal. The helically wound outer conductor 130 may comprise a defect or a difference in conductance between a first helically wound conductor and a second helically wound conductor to generate a magnetic field (i.e. non-negligible magnetic field) outside the coaxial cable 104 when the first EM signal is conveyed by the coaxial cable 104. This produces a magnetic field outside the helically wound outer conductor 130, and the magnetic field can be used for inductive power transfer from the transmitter 105 to the receiver 120.

The receiver 120 has a magnetizable element 122, and a receiving conductor 124 in which a current is generated. In this embodiment, the magnetizable element 122 is a ferrite core, and the receiving conductor 124 is a secondary winding. The receiver 120 inductively couples to the magnetic field produced by the coaxial cable 104, and therefore ends 126 can be connected to an electrical element or secondary circuit.

The coaxial cable 104 does not generate a non-negligible or useful magnetic field outside the helically wound outer conductor 130 when a second EM signal having a higher frequency is conveyed by the coaxial cable 104. The second EM signal may be a MW signal. Due to the second EM signal having a higher frequency, the skin depth is lower, and the second EM signal is conveyed across defects in the helically wound conductor (e.g. along a longitudinal path) due to the higher capacitive susceptance experienced by the second EM signal compared to the first EM signal (e.g. the defect capacitive susceptance for the MW signal may be 10, 000 times larger compared to the defect capacitive susceptance for the RF signal). As such, the helically wound outer conductor 130 will not leak a significant, e.g. non-negligible or useful, magnetic field when conveying the second EM signal. Therefore, the receiver 120 will not couple to the transmitter 105 when a second EM signal is conveyed by the coaxial cable 104.

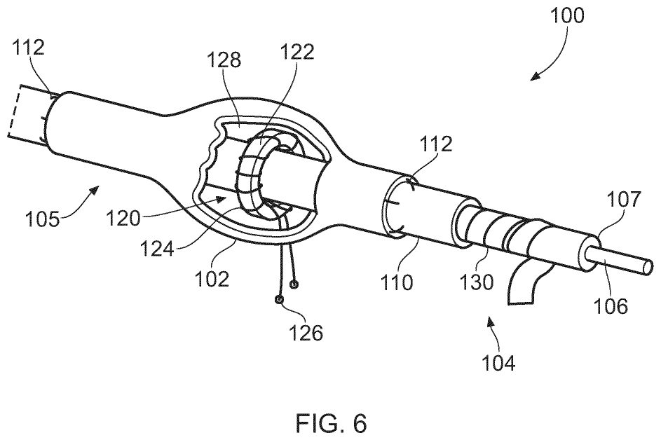

FIG. 6 shows a schematic diagram of a power transfer assembly 100 with an auxiliary conductor 102 and a helically wound outer conductor 130 according to a third embodiment. In this embodiment, the transmitter 105 comprises both the helically wound outer conductor 130 and the auxiliary conductor 102. Having both the helically wound outer conductor 130 and the outer conductor 102 generates a stronger magnetic field for the receiver 120 to couple to.

The auxiliary conductor 102 is coupled to the helically wound outer conductor 130 at electrical contacts 112. However, this connection may be capacitive in other embodiments. An intermediate portion 116 of the auxiliary conductor 102 is spaced from the helically wound outer conductor 130 to define a void 128. A receiver 120 is located within this void, and a receiving conductor 124 of the receiver 120 is connectable at ends 126 to a secondary power circuit or electrical element.

The helically wound outer conductor 130 runs through the area that is coupled to by the auxiliary conductor 102. When the helically wound outer conductor 130 conveys a first EM signal, e.g. an RF signal, the helically wound outer conductor 130 is leaky and thus generates a magnetic field outside the coaxial cable 104.

Further, when the first EM signal is conveyed by the helically wound outer conductor 130, a portion of the first EM signal will be flowing near the outer surface of the helically wound outer conductor 130. For example, the thickness and/or material of the helically wound outer conductor 130 is selected based on the skin depth of the first EM signal to cause a portion of the current to flow near the outer surface of the helically wound outer conductor 130. Therefore, at least part of the first EM signal will flow through the auxiliary conductor 102.

Therefore, as the coaxial cable 104 leaks due to the helically wound outer conductor 130, the signal flowing down the helically wound outer conductor 130 does not cancel out the magnetic field produced by the signal flowing down the inner conductor 106. This generates a magnetic field (e.g. useful or non-negligible magnetic field) outside the coaxial cable 104. Furthermore, a portion of the first EM signal flowing near the outer surface of the helically wound outer conductor 130 will flow down the auxiliary conductor 102, further amplifying the difference between the signal flowing down the inner conductor 106 and the helically wound outer conductor 130. This causes an even greater magnetic field outside the outer surface of the helically wound outer conductor 130. This magnetic field is coupled to by the receiver 120.

When the second EM signal, e.g. a MW signal, is conveyed by the coaxial cable 104, the coaxial cable 104 is not configured to create a magnetic field (i.e. useful or non-negligible magnetic field) outside the coaxial cable 104. This is because due to the higher frequency of the second EM signal, a negligible portion of the second EM signal flows near the outer surface of the helically wound outer conductor 130. Furthermore, due to the increased capacitive susceptance and the decreased skin depth, the helically wound outer conductor 130 does not leak significantly when conveying the second EM signal.

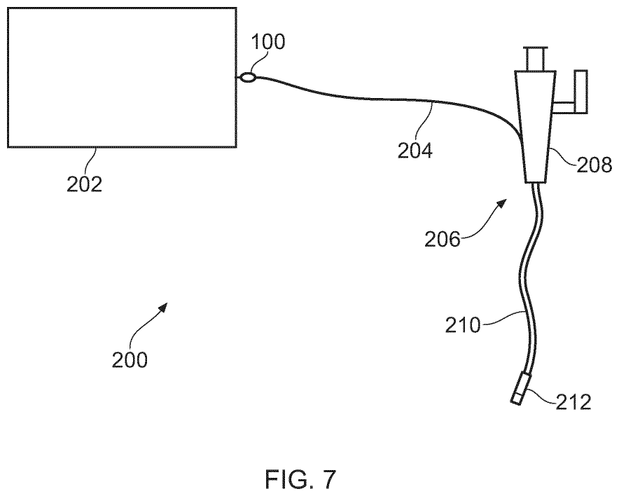

FIG. 7 shows an electrosurgical system 200 comprising a power transfer assembly 100 according to a fourth embodiment. The electrosurgical system 200 delivers EM energy to biological tissue for tissue treatment.

The electrosurgical system 200 comprises an energy source 202 which generates EM signals. For example, energy source 202 may be a generator which generates RF and MW signals. The electrosurgical system 200 further comprises a feed structure 204 for conveying EM energy from the energy source 202 to an applicator 212 located at a distal end of the feed structure 204. The feed structure 204 and the applicator 212 form at least part of an electrosurgical instrument.

The electrosurgical instrument may be for a surgical scoping device 206. For example, the electrosurgical instrument may be dimensioned to fit within the surgical scoping device 206. In this embodiment, the surgical scoping device 206 comprises a body 208 and an instrument chord 210. The instrument chord 210 may be configured to be insertable into a patient's body. The feed structure 204 extends through the instrument chord 210 with the applicator 212 located at a distal end of the feed structure 204 and the instrument chord 210, for delivery of EM energy into tissue. The instrument may be conveyed within an instrument channel or lumen within the instrument chord 210.

The electrosurgical system 200 comprises the power transfer assembly 100. Embodiments of the power transfer assembly 100 are shown in FIGS. 1 to 6. The feed structure 204 comprises the coaxial cable 104 of the transmitter 105. For example, the feed structure 204 may be a coaxial cable, and a section of the coaxial cable of the feed structure may be the coaxial cable 104 of the transmitter 105. The coaxial cable 104 of the transmitter may be located at any point along the feed structure 204. In this embodiment, the power transfer assembly 100 is located at a proximal end region of the feed structure 204, e.g. near where it joins to the energy source 202.

When a first EM signal is conveyed along the feed structure 204, and when the first EM signal reaches the power transfer assembly 100, a magnetic field will be generated outside the feed structure 204. This can then be coupled to by a receiver 120 of the power transfer assembly 100, to transfer energy via inductive coupling from the feed structure 204 to the receiver 120. In this embodiment the receiver 120 is located around the feed structure 204. However, in other embodiments the receiver may be located in a separate structure around the feed structure 204. When the second EM signal is conveyed along the feed structure 204, zero or near zero magnetic field is generated outside the feed structure 204, as explained previously.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

1. A power transfer assembly comprising:
a transmitter comprising a coaxial cable having an inner conductor and an outer conductor separated by a dielectric material, the coaxial cable being configured to radiate a magnetic field radially outside of the outer conductor when a first electromagnetic (EM) signal is conveyed by the coaxial cable; and
a receiver for receiving power from the transmitter by inductive coupling using the magnetic field.

2. The power transfer assembly according to claim 1, wherein the outer conductor comprises a first helically wound conductor.

3. The power transfer assembly according to claim 2, wherein the first helically wound conductor comprises a defect configured to cause a first magnetic field produced by the signal being conveyed by the outer conductor not to cancel out a second magnetic field produced by the signal being conveyed by the inner conductor, to thereby cause the coaxial cable to radiate the magnetic field radially outside of the outer conductor.

4. The power transfer assembly according to claim 3, wherein the defect increases resistance between adjacent windings of the first helically wound conductor to cause the first EM signal to follow a helical path along the first helically wound conductor in a region local to the defect.

5. The power transfer assembly according to claim 3, wherein the defect comprises a gap between adjacent windings of the first helically wound conductor.

6. The power transfer assembly according to claim 2, wherein the outer conductor further comprises a second helically wound conductor, and wherein the first helically wound conductor has a first electrical conductivity and the second helically wound conductor has a second electrical conductivity, the first electrical conductivity and the second electrical conductivity being different to radiate the magnetic field radially outside of the outer conductor.

7. The power transfer assembly according to claim 1, wherein the transmitter comprises an auxiliary conductor having a first portion and a second portion, each portion being electrically coupled to the outer conductor, the auxiliary conductor further having an intermediate portion between the first and second portions and which is radially spaced from the outer conductor to define a void, the auxiliary conductor and the outer conductor being configured to cause the first EM signal to flow through the auxiliary conductor.

8. The power transfer assembly according to claim 7, wherein the outer conductor has a thickness or material set according to a skin depth of the first EM signal being conveyed by the coaxial cable to cause the first EM signal to flow through the auxiliary conductor.

9. The power transfer assembly according to claim 7, wherein the outer conductor comprises a thinned section which is thinner than other sections of the outer conductor, wherein the auxiliary conductor is coupled to the thinned section, the thinned section having a thickness to cause the first EM signal to flow through the auxiliary conductor.

10. The power transfer assembly according to claim 7, wherein the first portion or the second portion is directly physically connected to the outer conductor.

11. The power transfer assembly according to claim 7, wherein the first portion or the second portion is spaced apart from the outer conductor to form a capacitive element for capacitively coupling the auxiliary conductor to the outer conductor.

12. The power transfer assembly according to claim 7, wherein the receiver is located in the void.

13. The power transfer assembly according to claim 1, wherein the receiver comprises a magnetizable element.

14. The power transfer assembly according to claim 13, wherein a receiving conductor of the receiver is wound around the magnetizable element.

15. The power transfer assembly according to claim 13, wherein the magnetizable element comprises a toroid.

16. The power transfer assembly according to claim 1, wherein the outer conductor is configured not to radiate a magnetic field radially outside of the outer conductor when a second EM signal is conveyed by the coaxial cable, the second signal having a higher frequency than the first signal.

17. The power transfer assembly according to claim 16, wherein the second EM signal is a microwave (MW) signal.

18. The power transfer assembly according to claim 1, wherein the first EM signal is a radiofrequency (RF) signal.

19. An electrosurgical instrument for delivering electromagnetic (EM) energy to biological tissue for tissue treatment, comprising:
a feed structure and an applicator located at a distal end of the feed structure, the feed structure for conveying the EM energy from an energy source to the applicator, the applicator for delivering the EM energy into biological tissue at a treatment site; and
a power transfer assembly according to claim 1, wherein the feed structure comprises the coaxial cable of the transmitter and the EM energy comprises the first EM signal.

20. The electrosurgical instrument according to claim 19, wherein the electrosurgical instrument is sized for insertion through an instrument channel of a surgical scoping device.
